(19) United States
(12) Patent Application Publication
(10) Pub. No.: US 2019/0090529 A1
(43) Pub. Date: Mar. 28, 2019

(54) VIBRATION DROPLET FORMATION

(71) Applicant: BASF SE, Ludwigshafen am Rhein (DE)

(72) Inventors: Kathrin MEYER-BOEHM, Ludwigshafen am Rhein (DE); Thrandur HELGASON, Illertissen (DE); Karl KOLTER, Ludwigshafen am Rhein (DE); Walter DOBLER, Ludwigshafen am Rhein (DE); Christol STADAGER, Ludwigshafen am Rhein (DE); MIchael SCHOENHERR, Ludwigshafen am Rhein (DE); Katja KESTEN, Ludwigshafen am Rhein (DE); Nikolaus NESTLE, Ludwigshafen am Rhein (DE)

(21) Appl. No.: 16/081,538

(22) PCT Filed: Feb. 28, 2017
(86) PCT No.: PCT/EP2017/054586

§ 371 Date: Aug. 31, 2018

(30) Foreign Application Priority Data
Mar. 2, 2016 (EP) 16158251.5

(51) Int. Cl. A23P 10/30; A23L 33/15; A23L 33/12; A23L 33/105; A61K 31/201; A23L 33/125; A61K 9/107; A61K 9/16; A23L 33/16

(52) U.S. Cl. CPC A23P 10/30; A23L 33/15; A23L 33/12; A23L 33/105; A23V 2002/00; A23L 33/125; A61K 9/107; A61K 9/16; A23L 33/16; A61K 31/201

(57) ABSTRACT

The present invention relates to a method for producing particles containing carotenoid and/or vitamin and/or omega-3 fatty acids and/or phytosterols and/or conjugated linoleic acids, having a narrow particle size distribution and uniform spherical shape and density, and also to particles obtainable by this method and use thereof as food supplements, foodstuffs, feedstuffs, body care products and medicaments. The particles according to the invention exhibit improved storage stability compared to the prior art.

The present invention relates to a method for producing particles containing carotenoid and/or vitamin and/or omega-3 fatty acids and/or phytosterols and/or conjugated linoleic acids, having a narrow particle size distribution and uniform spherical shape and density, and also to particles obtainable by this method and use thereof as food supplements, foodstuffs, feedstuffs, body care products and medicaments. The particles according to the invention exhibit improved storage stability compared to the prior art.

The particles comprising carotenoid and/or vitamin produced by the method described in U.S. Pat. No. 4,522,743 have a high proportion of air inclusions, which can account for >40% cavity based on the total particle volume. By virtue of these air inclusions, the particles are mechanically less stable than solid particles. The particles may be damaged during further processing and result in worsening the product properties. In addition to the air inclusions, these particles are usually characterized by a broad particle size distribution, possibly paired with a rather non-uniform particle shape. Particularly by crushing the hollow spheres under mechanical stress, but also due to the broad particle size distribution and the fine dust content associated therewith and the non-uniform particle shape, these preparations have a large surface area which renders them sensitive to oxidative attack by oxygen on the carotenoids and vitamins present in the particles.

The object of the invention, therefore, is to provide a method which affords particles which do not possess these disadvantages of the prior art.

The object is achieved by a process in which spherical particles are produced by dispersing carotenoid and/or vitamin and/or omega-3 fatty acids and/or phytosterols and/or conjugated linoleic acids in a solution comprising hydrocolloid and droplets are produced from the dispersion formed by a vibration dropletization process and the droplets obtained are solidified and dried by evaporation of the solvent.

In the context of the invention, spherical signifies that the individual particles each have an aspect ratio of 1 to 1.2. “Aspect ratio” is the quotient of the largest and smallest particle diameter.

In the context of the present invention, a dispersion means both emulsions and suspensions.

In the vibration dropletization process, the nozzle and/or the dispersion and/or a reservoir vessel containing the dispersion and/or a feed line supplying the dispersion to the nozzle are excited by vibrations. The vibration exciter used may be a mechanical oscillator, magnetic inductive oscillator, a pneumatic oscillator, a piezoelectric transducer or an electroacoustic transducer. In this case, the vibration generator can act on the nozzle and/or feed line and/or on the reservoir vessel. There is also the possibility to treat the dispersion directly with ultrasound, for example using an electroacoustic transducer, or to excite directly using a vibrating baffle/plunger, in order to dropletize the dispersion exiting the nozzle to uniform droplets.

These dropletizing processes have the advantage that a monodisperse distribution of the spherical particles that have been formed is obtained.

The frequency that acts on the device or the dispersion is kept constant during the production process, wherein preferably excitation frequencies are used between 50 to 10 000 Hz, preferably in the range 100 to 5000 Hz and particularly preferably in the range 400-4000 Hz. The viscosity of the dispersions is ≥80 mPas at 40° C. Lastly, the diameter of the nozzle should be in the range between 50 and 1000 μm.

With these parameters, spherical particles can be generated with a narrow particle size distribution in which, depending on the frequency and the nozzle diameter, particles having a diameter between 100 and 1500 μm are achievable.

In this case, the method is preferably carried out such that spherical panicles are obtained having a particle size distribution of ≥75% in the range from 150 to 600 μm, preferably ≥85% in the range from 150 to 600 μm and particularly preferably ≥95% from 150 to 600 μm.

In particular, the method is characterized in that the polydispersity, measured as the span of the particle size distribution (X90−X10 divided by X50), is less than 1.0, preferably less than 0.8 and particularly preferably less than 0.6, wherein the X50 value represents the mean particle size distribution and the difference X90−X10 represents the breadth of the particle size distribution.

The content of the spherical particles consists of a hydrocolloid matrix in which carotenoid(s) and/or vitamin(s) and/or omega-3 fatty acids and/or phytosterols and/or conjugated linoleic acids are present homogeneously distributed. These particles are characterized in that the cavity volume enclosed in the particles is ≤40%, preferably ≤30% and particularly preferably ≤20% of the total volume of the particles.

In the context of the present invention, carotenoids and/or vitamins and/or omega-3 fatty acids and/or phytosterols and/or conjugated linoleic acids are understood in this case to mean vitamins A, D, E or K or derivatives thereof, for example esters of vitamin A and vitamin E such as retinyl acetate or tocopherol acetate, tocotrienol, vitamin K1, vitamin K2, and also carotenoids such as R-carotene, canthaxanthin, astaxanthin, citranaxanthin and ester derivatives, zeaxanthin and ester derivatives, lutein and ester derivatives, lycopene and apocarotenal.

Suitable hydrocolloids in accordance with the invention are plant gums, modified plant gums, gelatine, modified gelatine, modified starch, lignosulfonate, chitosan, carrageenan, casein, caseinate, whey protein, zein, modified cellulose, pectin, modified pectin, plant proteins and modified plant proteins or mixtures thereof.

The plant gums include in this case agar, alginic acid, alginate, chicle, dammar, marshmallow extracts, gellan, guar seed meal, gum arabic, gum from plantain seed husk, gum from spruce tree sap, carob seed flour, karaya, konjac flour, mastic, tara bean gum, iragacanth, xanthan.

In accordance with the invention, preferred as hydrocolloid are gelatine and/or plant gums and/or modified plant gums and particularly gum arabic among the plant gums.

Furthermore, before or after addition of the carotenoid and/or vitamin and/or omega-3 fatty acids and/or phytosterols and/or conjugated linoleic acids, an antioxidant can be added to the hydrocolloid solution to increase the stability of the same against oxidative degradation. The antioxidant in this case is selected from the group consisting of dl-α-tocopherol, d-α-tocopherol, β-tocopherol, γ-tocopherol, δ-tocopherol, butylhydroxytoluene (BHT), butylhydroxyanisole, propyl gallate, octyl gallate, dodecyl gallate, extracts of rosemary, extracts of green tea and other gallic acid derivatives, tert-butylhydroxyquinoline, ethoxyquin, carnosol, carnosic acid, ascorbyl palmitate and ascorbyl stearate or mixtures thereof.

The proportion of antioxidants in the particle composition is 0.1 to 10% by weight, preferably 0.5 to 8.5% by weight, based on the dry mass of the particle composition (without powdering agent) comprising carotenoid and/or vitamin and/or omega-3 fatty acids and/or phytosterols and/or conjugated linoleic acids, wherein the sum total of the percentages of the individual components adds up to 100%.

To increase the mechanical stability of the spherical particles, it is also appropriate to add a softener to the hydrocolloid, such as sugars or sugar alcohols, e.g. sucrose, glucose, glucose syrup, lactose, invert sugar and other glucose-fructose compositions, sorbitol, mannitol, glycerol, maltodextrins, isomaltose or isomalt. The name isomalt denotes a sugar replacer which is also supplied under the tradename Palatinit® (Südzucker, Germany). Isomalt is a hydrogenated isomaltulose, which consists of approximately equal portions of 6-O-α-D-glucopyranosyl-D-sorbitol and 1-O-α-D-glucopyranosyl-D-mannitol. Softeners preferably used are sucrose, glucose syrup, sorbitol and lactose.

The ratio of protective colloid and softener to carotenoid and/or vitamin and/or omega-3 fatty acids and/or phytosterols and/or conjugated linoleic acids is generally selected so that a solid preparation is obtained comprising between 0.01 and 50% by weight carotenoid and/or vitamin, 10 to 65% by weight, preferably 15 to 60% by weight of a protective colloid and 5 to 60% by weight, preferably 10 to 50% by weight of a softener, wherein all percentages refer to the dry mass of the particle composition (without powdering agent) and the sum total of the percentages of the individual components adds up to 100%.

Furthermore, emulsifiers may be used, for example ascorbyl palmitate, polyglyceryl fatty acid esters, sorbitan fatty acid esters, propylene glycol fatty acid esters or lecithin, at a concentration of 0 to 200% by weight, preferably 5 to 150% by weight, particularly preferably 10 to 80% by weight, based on the carotenoid(s) and/or vitamin(s) used.

It can also be possibly advantageous to use in addition a physiologically tolerable oil such as, for example, sesame oil, corn germ oil, cottonseed oil, soybean oil, peanut oil, sunflower oil, rapeseed oil, coconut oil, palm oil, olive oil, animal fats, lard, tallow, modified oils or mixtures thereof at a concentration of 0 to 500% by weight, preferably 10 to 300% by weight, particularly preferably 20 to 100% by weight, based on the carotenoid(s) and/or vitamin(s).

In addition to the constituents specified, other customary auxiliaries and additives, such as inorganic and organic salts, may be advantageously added to the dispersion for the preparation of particle compositions containing carotenoid and/or vitamin and/or omega-3 fatty acids and/or phytosterols and/or conjugated linoleic acids.

The proportion of auxiliaries and additives is generally 0.2 to 20% by weight, preferably 0.3 to 15% by weight, particularly preferably 0.4 to 10% by weight, especially preferably 0.5 to 5% by weight, based on the dry mass of the particle composition (without powdering agent), wherein the sum total of the percentages of the individual components adds up to 100%.

A pulverulent preparation can be made from the dispersion in a manner known per se in the fluidized bed, in accordance with the details in DE2534091 for example, by spray drying or by spray cooling or by enveloping the particles, separating and drying.

The droplets formed in this case are enveloped by a powdering agent which may be selected from hydrophobic silica, hydrophilic silica, starch, modified starch, corn starch, celluloses, modified celluloses, calcium silicate, calcium-magnesium silicate, calcium carbonate, tricalcium phosphate, calcium adipate, magnesium adipate, titanium dioxide, lignins, highly dispersed pectin, modified pectin, plant proteins, modified plant proteins and combinations of these.

The method according to the invention is characterized in that the droplets generated are coated with a powdering agent at temperatures between 10 and 80° C. and are subsequently solidified and dried at feed air temperatures between 40 and 120° C.

Preference is given to a process regime in which a dispersion comprising carotenoid and/or vitamin and/or omega-3 fatty acids and/or phytosterols and/or conjugated linoleic acids is sprayed into an inert gas atmosphere laden with the hydrophobic silica or corn starch powdering agents.

In particular, preference is given to a process regime in which, after spraying, the powder is dried to a residual moisture content of below 10% by weight, preferably below 6% by weight.

The invention further relates to particle compositions which, in addition to the constituents specified, comprise 0.025-fold to 4-fold proportion by weight with respect to active ingredient of powdering agents or powdering agent mixtures. In addition, the use of the preparation forms according to the invention as food supplements, foodstuffs, feedstuffs, body care products and medicaments is claimed.

### FIGURES

FIG. 1: Scanning electron micrograph of the particles produced according to 1A

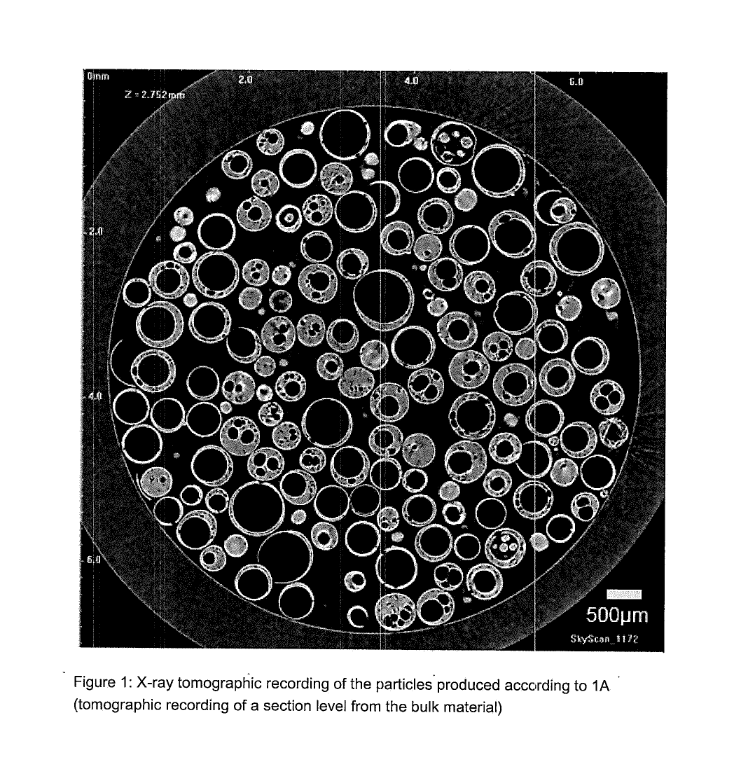

FIG. 2: Scanning electron micrograph of the particles produced according to 1B

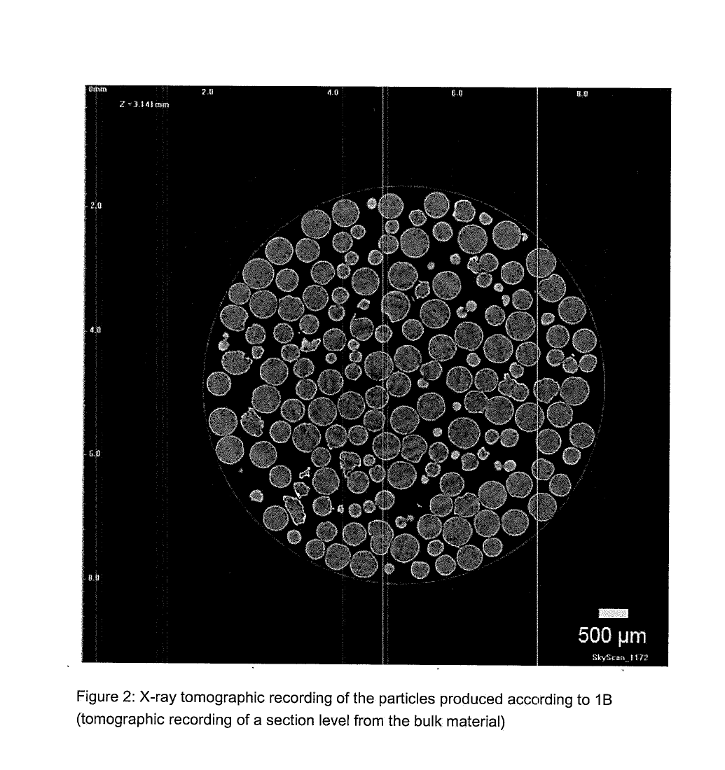

FIG. 3: Particle size distribution of experiments 1A and 1B

In the examples below, the preparation of the particles according to the invention is explained in more detail.

### NON-INVENTIVE EXAMPLE 1A

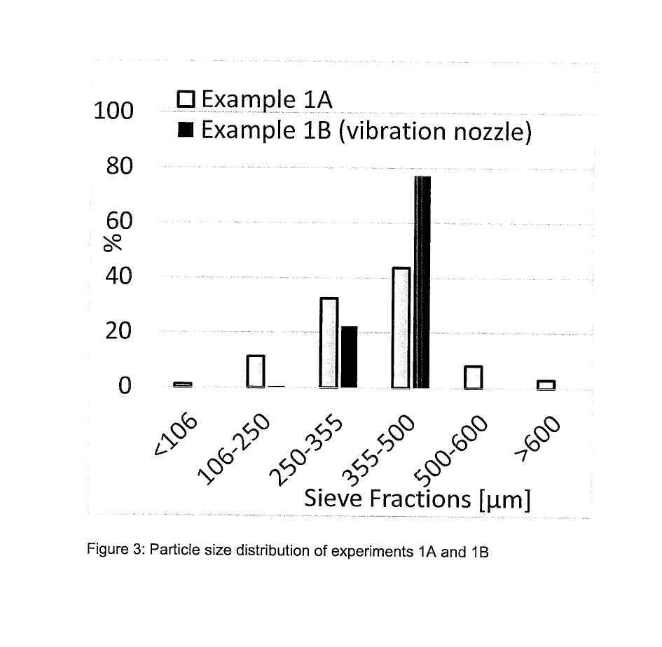

30 g of canthaxanthin are suspended in 240 g of isopropanol together with 0.6 g of ascorbyl palmitate and 8 g of ethoxyquin and, on setting the pressure limiting valve to 30 bar, mixed continuously with 390 g of isopropanol in a mixing chamber A. At a metering rate of 6 l/h on the suspension side and of 9 l/h on the solvent side, a mixing temperature of 170° C. is set in the mixing chamber A. After a residence time of 0.3 seconds, the molecularly disperse solution is mixed in mixing chamber B with a solution of 32 g of gelatine and 120 g of glucose syrup in 4000 g of water at a flow rate of 100 I/h of isopropanol. After removal of the solvent under reduced pressure in a distillation apparatus, an active ingredient dispersion is obtained which can be converted by spray drying to a stable, water-soluble dry powder. After dissolution in water, a particle size of 150 nm is measured. The emulsion thus prepared was sprayed into a spray tower via a nozzle at 25 bar in which hydrophobic silica was fluidized at 60° C. The still moist particles were further dried at 60° C. air inlet temperature in the underlying fluidized bed for 5h. A broad particle size distribution (FIG. 3) was determined for the particles with a maximum at ca. 500 μm, associated with a high proportion of cavities, based on the total volume of the particles.

### Determination of the Proportion of Hollow Spheres in Example 1A

4 ml of the particles produced were charged in a 15 ml centrifuge tube. n-Pentane was then added to the tube until a volume of 12 ml had been reached. The tube was sealed and shaken until the particles had been completely stirred up from the bottom. The tube was then placed in an upright position and the measurement was assessed after 5 minutes.

The amount of hollow spheres was read off a mm scale and specified in millimeters.

For example 1A, a mean value of 6.2 mm was measured in a triplicate determination.

### INVENTIVE EXAMPLE 1B

Example 1B was carried out analogously to Example 1A with the difference that a vibration nozzle was used at a pressure of 0.5 bar. The device used was a Buchi Encapsulator B-390 with a 200 μm nozzle opening at a frequency of 1400 Hz. The flow rate achieved through a nozzle was 30 g per hour. A narrow particle size distribution (FIG. 3) was determined for the particles with a maximum at ca. 400 μm, associated with a very low proportion of cavities, based on the total volume of the particles.

### Determination of the Proportion of Hollow Spheres in Example 1B

The amount of hollow spheres of example 1B was determined according to the experimental method of example 1A.

For example 1B, a mean value of 0 mm was measured in a triplicate determination.

Stability Test for Canthaxanthin The stability of the particles thus produced was tested in a premix stress test. For this purpose, test specimens of 25 mg of the particles produced in each case and 4 g of premixed mixture was weighed into 50 ml glass bottles. The premixed mixture consisted of 20% wheat semolina bran, 20% of 50% choline chloride supported on silica and 10% trace element mixture. The trace element mixture consisted of 46.78% FeSO₄x7H₂O, 37.43% CuSO4x5H₂O, 11.79% ZnO, 3.61% MnO and 0.39% CoCO₃. After addition of all ingredients, the test specimens were carefully mixed by hand. These test specimens were stored in a climate chamber at 40° C. and 70% for 4 weeks. Prior to commencement of the storage and after completion of the storage, the canthaxanthin content of the test specimens was determined. From the ratio of the canthaxanthin contents after and prior to storage, the retention was calculated.

The retention values of the examples are compiled in the table which follows.

| Name | Retention (%) |
|---|---|
| 1A | 25 |
| 1B | 86 |

The higher the retention, the better the stability of the particles or preparation thereof. If the stability of the particles of the inventive example is compared to the corresponding non-inventive example, the improvement in the stability is clearly apparent.

1.-18. (canceled)

19. A method for producing spherical particles containing carotenoid and/or vitamin and/or omega-3 fatty acids and/or phytosterols and/or conjugated linoleic acids, wherein carotenoid and/or vitamin and/or omega-3 fatty acids and/or phytosterols and/or conjugated linoleic acids are dispersed in a solution comprising at least one hydrocolloid and droplets are produced from the dispersion formed by means of a nozzle, wherein the dispersion droplets are generated by vibrational excitation and the droplets are solidified and dried by evaporation of the solvent.

20. The method according to claim 19, wherein the nozzle and/or the dispersion and/or a reservoir vessel containing the dispersion and/or a feed line supplying the dispersion to the nozzle are excited by vibrations.

21. The method according to claim 19, wherein the vibrational excitation is a superimposed frequency of vibration in the range from 50 to 10 000 Hz.

22. The method according to claim 19, wherein the dispersion to be dropletized has a viscosity of ≥80 mPas at 40° C.

23. The method according to claim 19, wherein carotenoids and/or vitamins and/or omega-3 fatty acids and/or phytosterols and/or conjugated linoleic acids are homogeneously distributed in the hydrocolloid matrix of the particle.

24. The method according to claim 19, wherein the cavity volume enclosed in the particles is ≤40% of the total volume of the particles.

25. The method according to claim 19, wherein the particles have a particle size distribution greater than 75% in the range from 150 to 600 μm.

26. The method according to claim 19, wherein the polydispersity, measured as X90−X10 divided by X50, is less than 1.0.

27. The method according to claim 19, wherein the hydrocolloid is selected from the group consisting of plant gums, modified plant gums, gelatine, modified gelatine, modified starch, lignosulfonate, chitosan, carrageenan, casein, caseinate, whey protein, zein, modified cellulose, pectin, modified pectin, plant proteins and modified plant proteins and mixtures thereof.

28. The method according to claim 19, wherein the carotenoid and/or vitamin and/or omega-3 fatty acids and/or phytosterols and/or conjugated linoleic acids is selected from the group consisting of vitamins A, D, E, K, derivatives thereof, and mixtures thereof.

29. The method according to claim 19, wherein the dispersion to be dropletized comprises at least one antioxidant selected from the group consisting of dl-α-tocopherol, d-α-tocopherol, β-tocopherol, γ-tocopherol, δ-tocopherol, butylhydroxytoluene (BHT), butylhydroxyanisole, propyl gallate, octyl gallate, dodecyl gallate, extracts of rosemary, extracts of green tea and other gallic acid derivatives, tert-butylhydroxyquinoline, ethoxyquin, carnosol, carnosic acid, ascorbyl palmitate and ascorbyl stearate and mixtures thereof.

30. The method according to claim 19, wherein the dispersion to be dropletized comprises an oil selected from the group consisting of sesame oil, corn germ oil, cottonseed oil, soybean oil, peanut oil, sunflower oil, rapeseed oil, coconut oil, palm oil, olive oil and animal fats, lard and tallow, modified oils and mixtures thereof.

31. The method according to claim 19, wherein the dispersion to be dropletized comprises at least one softener selected from sugars or sugar alcohols.

32. The method according to claim 19, wherein the droplets generated by vibrational excitation are coated with a powdering agent and are subsequently solidified and dried.

33. The method according to claim 19, wherein the droplets generated are coated with a powdering agent at temperatures between 10 and 80° C. and are subsequently solidified and dried at feed air temperatures between 40 and 20° C.

34. The method according to claim 32, wherein the powdering agent is selected from the group consisting of hydrophobic silica, hydrophilic silica, starch, modified starch, corn starch, celluloses, modified celluloses, calcium silicate, calcium-magnesium silicate, calcium carbonate, tricalcium phosphate, calcium adipate, magnesium adipate, titanium dioxide, lignins, highly dispersed pectin, modified pectin, plant proteins, modified plant proteins and combinations of these.

35. A preparation form obtained by the method according to claim 19.

36. A food supplement, foodstuff, feedstuff, body care product, or medicament comprising the preparation form according to claim 35.

37. The method according to claim 19, wherein the vibrational excitation is a superimposed frequency of vibration in the range from 100 to 5000 Hz.

38. The method according to claim 19, wherein the vibrational excitation is a superimposed frequency of vibration in the range from 400 to 4000 Hz.
